(19) United States
(12) Patent Application Publication
(10) Pub. No.: US 2014/0079931 A1
(43) Pub. Date: Mar. 20, 2014

(54) CELLULOSE-BASED MATERIALS COMPRISING NANOFIBRILLATED CELLULOSE FROM NATIVE CELLULOSE

(71) Applicants: Lars Berglund, Akersberga (SE); Houssine Sehaqui, Dubendorf (CH); Qi Zhou, Taby (SE)

(72) Inventors: Lars Berglund, Akersberga (SE); Houssine Sehaqui, Dubendorf (CH); Qi Zhou, Taby (SE)

(73) Assignee: CELLUTECH AB, STOCKHOLM (SE)

(21) Appl. No.: 14/007,604

(22) PCT Filed: Mar. 26, 2012
(86) PCT No.: PCT/SE12/50332

§ 371 Date: Dec. 6, 2013

Related U.S. Application Data
(60) Provisional application No. 61/467,450, filed on Mar. 25, 2011.

(30) Foreign Application Priority Data
Apr. 4, 2011 (SE) 1150292-9

(51) Int. Cl. C08B 16/00; C08L 1/02; C12P 19/14

(52) U.S. Cl. CPC C08B 16/00; C12P 19/14; C08L 1/02
USPC 428/219; 536/56; 435/99

(57) ABSTRACT

The present invention relates to cellulose-based materials comprising nanofibrillated cellulose (NFC) from native cellulose. exhibiting highly superior properties as compared to other cellulose-based materials, a method for preparing such cellulose-based material, and uses thereof are also disclosed.

### TECHNICAL FIELD

The present invention relates to cellulose-based materials, for instance membranes, nanoporous solids, aerogels, and nanopapers, comprising nanofibrillated cellulose (NFC), methods for preparing said cellulose-based materials, as well as various uses of said cellulose-based materials.

### TECHNICAL BACKGROUND

Numerous biopolymers exhibit appealing characteristics for many industrial applications, for instance within the paper and textile industries but also within various types of separation processes, as well as within polymer and paint, pharmaceutical, and biomedical industries. Cellulose is a highly abundant and extensively characterized biopolymer of great significance not only as a basis for paper and textile manufacture but cellulose-based materials are increasingly employed for applications within fuel cell technology, liquid purification and filtering, tissue engineering, protein immobilization and separation, protective clothing, permeation and adsorption, heat and acoustic insulation, electrodes, optical applications, carriers for catalysis or drug delivery/release, or composite materials.

Most technical applications of cellulose-based materials require that cellulose is regenerated, i.e. physically altered, from its native state. Regeneration of cellulose involves dissolving the polysaccharide using various types of ion-containing organic solvents (such as DMAC/LiCl, N-methylmorpholine-N-oxide (NMMO), sodium hydroxide (NaOH)/urea, NaOH/thiourea) followed by precipitation in aqueous solution. Consequently, in addition to being time-consuming and costly, the need for cellulose regeneration implies that an otherwise ‘green’ alternative becomes less advantageous from an environmental point of view, and, furthermore, the essentially irreversible nature of the conversion of native cellulose (cellulose type I) into regenerated cellulose (cellulose type II) means that the useful properties of native cellulose are lost.

The utility of cellulose-based material, be it in fuel cells, as nanopaper, or for permeation and adsorption purposes, is contingent upon parameters such as mechanical strength, chemical inertness, porosity, pore diameter, and large specific surface area. Thus, the ability to successfully control these parameters during production of the material as such is highly important.

The prior art contains several disclosures describing the use of regenerated cellulose for the preparation of for instance cellulose aerogels, nanopapers, and membranes with conceivable usefulness as adsorbents, heat/sounds insulators, filters or catalyst supports. Cai and co-workers (ChemSusChem, 2008, 1, 149-154) have, for example, described the preparation of cellulose aerogels through a procedure comprising regeneration of cellulose (using acids, alcohols, or acetone) into films of cellulose type II, followed by solvent exchange to ethanol, and finally conventional freeze-drying or supercritical CO₂ (sc-CO₂) drying.

Specific surface area (SSA) has been reported for different cellulose-based materials. Aerogel and foam materials based on freeze-dried NFC have data of 20-66 m²/g and 10-40 m²/g respectively. Aerogels from regenerated cellulose (dissolved and precipitated) can have a specific surface area of 500 m²/g (Cai, et al.) when prepared by sc-CO₂, but the structure of the aerogel is not a fibrous network.

### SUMMARY OF THE INVENTION

It is consequently an object of the present invention to overcome the drawbacks within the art relating to the need for regeneration of cellulose. Further, through for the first time enabling the use of native cellulose (cellulose type I) in methods for preparing cellulose-based materials, the present invention provides simplified, fast, and more environmentally friendly methods for preparing such materials, as well as the cellulose-based materials per se comprising cellulose type I. Additionally, the present invention allows for enhanced utility of cellulose-based materials, in part as a result of the improved control of the preparation method and partly as an implication of the advantages fact that cellulose type I can be utilized,

More specifically, the present invention relates to cellulose-based materials, for instance membranes, nanoporous solids, aerogels, and/or nanopapers, comprising nanofibrillated cellulose (NFC) present as cellulose type I, methods for preparing said superior cellulose-based materials, as well as various uses of said cellulose-based materials in the contexts of fuel cells, liquid purification and filtering, tissue engineering, protein immobilization and separation, protective clothing, permeation and adsorption, heat and acoustic insulation, electrodes, optical applications, carriers for catalysis or drug delivery/release, or composite materials. The methods in accordance with the present invention enable rapid, scalable, and robust preparation of cellulose-based materials with inter alia enhanced mechanical and physical properties.

One object of the present invention relates to a cellulose-based material comprising NFC from native cellulose, wherein the cellulose-based material comprises NFC in the form of cellulose type I. Another object of the present invention is a cellulose-based material obtainable through the methods as per the present invention.

Another object of the present invention is a method for preparing a cellulose-based material from native cellulose. The cellulose-based material comprises nanofibrillated cellulose (NFC) in the form of cellulose type I (i.e. crystalline cellulose), and the method comprises the steps of: (a) obtaining a hydrogel comprising NFC in the form of cellulose type I, (b) substantially exchanging the solvent of the NFC dispersion at least once for at least one second solvent, and (c) removing the at least one second solvent by at least one of (i) liquid evaporation and (ii) supercritical drying

A further object of the present invention is the use of the cellulose-based materials as nanoporous solids, aerogels, nanopapers, and/or membranes, for instance in the contexts of fuel cells, liquid purification and filtering, tissue engineering, protein immobilization and separation, protective clothing, permeation and adsorption, heat and acoustic insulation, electrodes, optical applications, carriers for catalysis or drug delivery/release, or composite materials.

### BRIEF DESCRIPTION OF THE DRAWINGS

FIG. 1 shows images of TO-NFC dispersion (a), a TO-NFC hydrogel (b), and of a typical porous NFC nanopaper (c).

FIG. 2 shows the pore size distribution of nanopaper based on BJH analysis. NFC nanopaper (left) and TO-NFC nanopaper (right). Data are for three different preparation routes; supercritical CO₂ drying (SC—CO₂), liquid CO₂ evaporation (L-CO₂) and tert-butanol freeze-drying (Tert-B-FD).

FIG. 3 plots average BJH pore diameter versus porosity for NFC nanopaper.

FIG. 4 shows FE-SEM images of (left, a) TO-NFC nanopaper prepared by SC—CO₂, SSA 482 m²/g (center, b) NFC nanopaper prepared by SC—CO₂, SSA 304 m²/g (surface of tensile fractured sample) and (right, c) NFC nanopaper prepared by Tert-B-FD, SSA 117 m²/g.

FIG. 5 shows tensile stress-strain curves for NFC nanopaper (left) and TO-NFC nanopaper (right). The different preparation methods and the corresponding porosities are provided.

FIG. 6 shows Young's modulus in tension (left) and tensile strength (right) as a function of relative density (ratio between nanopaper density and cellulose density). Relative density is equal to volume fraction.

FIG. 7 shows (a) folded NFC nanopaper prepared by SC—CO₂, (b) same nanopaper after 10 cycles of folding-unfolding, (c) TO-NFC nanopaper prepared by L-CO₂ on top of a logo in order to illustrate optical transparency.

FIG. 8 plots sorption isotherms of NFC aerogels. Porosity is in the range 98.5%-99.1%.

FIG. 9 shows SEM micrographs of a surface (a) and cross-section (b) of an NFC aerogel with a density of 30 kg/m³.

FIG. 10 shows compression stress-strain curves of (a) NFC aerogels and (b) NFC foams. Numbers next to each curve represent density values in kg/m³. Magnified sections in upper left corners show (a) strain hardening behaviour of the NFC aerogels and (b) yield behaviour of the NFC foams.

FIG. 11 shows modulus E* as a function of relative density ρ*/ρₛ (=volume fraction of solid material) for NFC aerogels and foams. ρ* is density of porous material, ρₛ is density of solid material.

FIG. 12 shows modulus as a function of density for the most common aerogels. Data for aerogels in the upper left corner are taken from Reichenauer G. Aerogels. In: John Wiley & Sons I, editor. Kirk-Othmer Encyclopedia of Chemical Technology. Data for cellulose aerogels in the lower right corner are taken from the present study. Native means NFC-based with the same crystal structure as in plants (cellulose I). Regenerated means dissolved cellulose, which is precipitated in suitable liquid (regenerated) so that a solid network with cellulose II structure (similar to Viscose) is formed.

FIG. 13 plots energy absorption versus relative density (ρ*/ρₛ) for NFC aerogel and other reported cellular materials. ρₛ of polystyrene is taken as 1050 kg/m³. ρₛ of epoxy/clay aerogel is calculated by taking 2860 kg/m³ as the density of clay (Cloisite Na, Southern Clay) and 1250 kg/m³ as the density of epoxy.

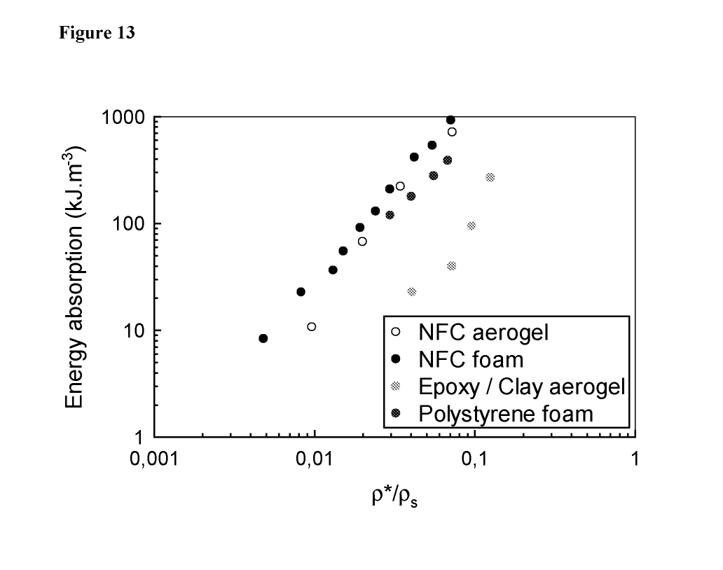

FIG. 14 plots stress-strain curves in compression of NFC aerogel network and NFC foam. Density is shown next to the curve.

### DETAILED DESCRIPTION OF THE INVENTION

The present invention relates to cellulose-based materials, for instance membranes, nanoporous solids, aerogels, and nanopapers, comprising nanofibrillated cellulose (NFC) present as cellulose type I, methods for preparing said cellulose-based materials, as well as various uses of said cellulose-based materials.

Where features, embodiments, or aspects of the present invention are described in terms of Markush groups, a person skilled in the art will recognize that the invention may also thereby be described in terms of any individual member or subgroup of members of the Markush group. The person skilled in the art will further recognize that the invention may also thereby be described in terms of any combination of individual members or subgroups of members of Markush groups. Additionally, it should be noted that embodiments and features described in the context of one of the aspects and/or embodiments of the present invention may also apply mutatis mutandis to all the other aspects and/or embodiments of the invention. For instance, features described in connection with the liquid evaporations step may naturally also apply mutatis mutandis in the context of the supercritical drying step, and the features described in connection with the cellulose-based material as such may naturally also apply mutatis mutandis in the context of the method for preparing said cellulose-based materials, all in accordance with the present invention as such.

All words and abbreviations used in the present application shall be construed as having the meaning usually given to them in the relevant art, unless otherwise indicated. For clarity, some terms are however specifically defined below.

As will be apparent from the description and the examples, the term “hydrogel” shall be understood to pertain to a network of hydrophilic polymer with water or any other type of aqueous solution as the dispersion medium, and the term “organogel” shall be understood to relate to a network entrapping a liquid phase comprising organic solvents, such as alcohols and in the context of the present invention also CO₂, etc. In the present context “solvent” refers only to the liquid used, but native cellulose is never dissolved in the procedure. Further, the term “enzymatic treatment” shall be understood to encompass all forms of exposure of cellulose to one or more enzymes (for instance endo- and/or exoglucanases, cellulases, etc.) having the capacity to catalyze at least one chemical reaction, the term “mechanical treatment” shall be understood to relate to exposing cellulose to any form of mechanical forces, whereas the term “chemical treatment” shall be understood to pertain to exposing cellulose to any form of chemical process and/or reaction, for instance oxidation, carboxymethylation, acid treatments, base treatments, etc. Additionally, the term “native cellulose” shall be understood to relate to cellulose with the same crystal structure as in plants (i.e. cellulose type I), whereas regenerated cellulose means dissolved cellulose, which is precipitated in suitable liquid (i.e. cellulose type II). With respect to fiber dimensions, the terms “diameter” and “thickness” are used interchangeably throughout the specification. The term “diameter” shall be understood to relate to the thickness of the fiber, irrespectively of whether the cross-section of the fiber is perfectly circular or not. The term “at least”, when used in a context such as “a strain-to-failure of at least 20%”, shall be understood to imply that the strain-to-failure in this case may range from at least approximately 20% to 100%. The feature “substantially exchanging” used in connection with solvent and/or solution exchange shall be understood to pertain to replacing a major part (i.e. >50%, but preferably an even larger proportion) of a first solvent/solution with a second solvent/solution.

One object of the present invention relates to a cellulose-based material comprising NFC from native cellulose, wherein the cellulose-based material comprises NFC in the form of cellulose type I, wherein the material has a specific surface area (SSA) of at least 200 m²/g, and a nanofiber network structure, wherein the nanofibers have a diameter less than 40 nm.

In one embodiment as per the present invention, the cellulose-based material may comprise NFC in the form of cellulose type I having a thickness less than 40 nm, preferably in the range of approximately 2-40 nm, more preferably 2-20 nm, even more preferably 3-10 nm. The length of the NFC nanofibers as per the present invention may range from approximately 50 nm to a few centimetres, but the cellulose type I-NFC length will most often be in the μm to mm range, naturally depending on the intended use of the cellulose-based material. The size of the NFC nanofibers influence the properties of the resulting cellulose-based material significantly, meaning that optimizing the thickness, the length, and/or the aspect ratio is crucial in order to successfully control the formation and the properties of the cellulose-based materials as per the present invention. Further, nanofiber network structure and random-in-the-plane NFC orientation distribution contributes significantly to the superior properties of the cellulose-based materials as per the present invention, as does the fact that the NFC are present in the form of cellulose type I (and not cellulose type II).

In a further embodiment, the cellulose-based material may have a specific surface area of at least 200 m²/g, preferably at least 300 m²/g, and more preferably at least 400 m²/g. The specific surface area, i.e. the how much exposed area the cellulose-based material has, is of great importance for inter alia chemical kinetics, such as in membrane, chromatography, and/or purification processes. The cellulose-based NCF-containing material as per the present invention displays an unusually high specific surface area, rendering the material superior to previously disclosed cellulose-based materials of the prior art.

The nanopaper structures of the present invention, which do not rely on cellulose dissolution, have a highly homogeneous nanofiber network structure, without regions of aggregated NFC.

Another object of the present invention relates to a method for preparing a cellulose-based material from native cellulose according to the present invention. The cellulose-based material comprises nanofibrillated cellulose (NFC) in the form of cellulose type I (i.e. crystalline cellulose), and the method comprises the steps of: (a) obtaining a hydrogel comprising NFC in the form of cellulose type I, (b) substantially exchanging the solvent of the NFC dispersion at least once for at least one second solvent, and (c) removing the at least one second solvent by at least one of (i) liquid evaporation and (ii) supercritical drying. In one embodiment, the dispersion of step (a) may be an aqueous dispersion, and, in a further embodiment, the at least one second solvent may be a water-miscible solvent.

Removing the at least one second solvent in step (c) by at least one of (i) liquid evaporation and (ii) supercritical drying, instead of using freeze-drying for the removal of the solvent, gives a cellulose-based material from native cellulose with a higher specific surface area as is demonstrated in the disclosed examples.

In one embodiment, the present invention pertains to a method for preparing a cellulose-based material comprising nanofibrillated cellulose (NFC). The method comprises the steps of: (a) obtaining a hydrogel comprising NFC in the form of cellulose type I by disintegrating native cellulose, (b) obtaining an organogel (comprising the NFC in the form of cellulose type I) by substantially exchanging the solvent of the hydrogel of the previous step at least once for at least one water-miscible solvent, and (c) removing the at least one water-miscible solvent by at least one of liquid evaporation and supercritical drying. Importantly, the present invention does not require cellulose to be regenerated prior to carrying out the method, which is a clear advantage over existing techniques within the technical field which all demand regeneration of the polysaccharide before preparation of cellulose-based materials (be it aerogels, nanopaper, membranes, and/or nanoporous solids) can be performed. Thus, the present invention represents a significant lead forward within the field of cellulose-based materials and their preparation, as it enables the use of native cellulose (i.e. cellulose type I).

In one embodiment, step (a) may comprise enzymatic treatment and/or mechanical treatment and/or chemical treatment. In a further embodiment, the enzymatic treatment may comprise endoglucanase treatment, exoglucanase treatment, and/or cellulase treatment. In yet another embodiment in accordance with the present invention, the chemical treatment may comprise 2,2,6,6-tetramethylpiperidine-1-oxy radical (TEMPO) oxidation, carboxymethylation, acid treatment, and/or base treatment. It is worth noting that albeit that various forms of pretreatment procedures may be carried out on the native cellulose, it still remains in the form of type I cellulose, in contrast to other techniques employed within the art which entail regeneration into cellulose type II.

Further as per the present invention, step (a) may be divided into at least two sub-steps comprising (a1) disintegrating native cellulose into NFC in the form of cellulose type I and (a2) filtrating the NFC of step (a1) to obtain a hydrogel comprising the cellulose type I-NFC. The filtration procedure may comprise filtrating the disintegrated native cellulose, which may be present in the form of an aqueous dispersion, through a suitable filter, for instance a filter having a pore size of for instance around 0.5 μm, preferably around 0.65 μm. The NFC dispersion may be diluted to a concentration of between approximately 0.05 wt % and 3 wt % and/or degassed prior to the filtration procedure.

In yet another embodiment, step (b) may be divided into the sub-steps of (b1) substantially exchanging the solvent of the hydrogel of step (a) for a water-miscible organic solvent and (b2) mixing the water-miscible organic solvent with CO₂. The organic solvent may be selected from a group comprising at least one water-miscible alcohol, acetone or any other suitable organic solvent known to a person skilled in the art. The CO₂ may be present in liquid form, which can be achieved through pressurizing the CO₂ to a suitable pressure.

In a further embodiment, the liquid evaporation step and/or the supercritical drying step may be preceded by step (b) comprising substantially exchanging the liquid component of the NFC-containing hydrogel for an organic solvent followed by substantially exchanging the organic solvent for CO₂.

Further in accordance with the present invention, the cellulose-based material is a nanoporous solid, an aerogel, a nanopaper, and/or a membrane, or any other type of cellulose-based material comprising NFC in the form of cellulose type I. Consequently, the present invention is associated with numerous advantages, for instance as a result of the fact that the methods as per the present invention enables using native cellulose (cellulose type I), meaning that the present invention does not require cellulose to be regenerated (from its native form) into cellulose type II. The present invention thus concerns cellulose that is not regenerated, i.e. cellulose that is not present in the form of cellulose type II.

A further object of the present invention pertains to a cellulose-based material obtainable through the methods as per the present invention.

The mechanical properties of the cellulose-based NFC-containing material are naturally crucial for a number of uses within different technical fields. Consequently, in yet another embodiment, the cellulose-based material may have a modulus of at least 0.1 GPa, preferably at least 0.4 GPa, more preferably at least 1 GPa, even more preferably at least 5 GPa. The cellulose-based material may in accordance with a further embodiment have an ultimate strength of at least 0.5 MPa, preferably at least 1 MPa, more preferably at least 10 MPa, even more preferably at least 50 MPa, and in yet another embodiment, the cellulose-based material may have a strain-to-failure of at least 1%, preferably at least 5%, more preferably at least 20%.

The pore diameter of the cellulose-based material is an important property for various applications, for instance relating to filtration and membrane functionalities. Thus, the cellulose-based material may in one embodiment have an average pore diameter of at least 1 nm, preferably at least 5 nm, more preferably at least 10 nm, even more preferably at least 50 nm. The porosity is analogously of paramount importance for a cellulose-based NFC-containing material, and, in accordance with further embodiments as per the present invention, the cellulose-based material may have a porosity of at least 20%, preferably at least 40%, more preferably at least 60%, and even more preferably at least 70%, and most preferably at least 90%.

In one embodiment, the cellulose-based material may be present in the form of a nanoporous solid, an aerogel, a nanopaper, and/or a membrane. The present invention allows for tailoring the resulting cellulose-based material into various physical forms through optimizing the method for preparing the cellulose-based materials. A further object of the present invention is the use of the cellulose-based materials as nanoporous solids, aerogels, nanopapers, and/or membranes, for instance in the contexts of fuel cells, liquid purification and filtering, tissue engineering, protein immobilization and separation, and protective clothing, permeation and adsorption, heat and acoustic insulation, electrodes, optical applications, carriers for catalysis or drug delivery/release, or composite materials.

### EXPERIMENTAL SECTION

### Example 1

### Materials

TEMPO (2,2,6,6-Tetramethyl-1-piperidinyloxy, free radical), Sodium hypochlorite (NaClO) solution (reagent grade, available chlorine 10-15%) were purchased from sigma Aldrich and used as received.

The preparation procedure of the porous cellulose nanopapers may the following steps: NFC disintegration from wood pulp fibers in the form of a water dispersion, followed by hydrogel formation from NFC dispersion by a filtration procedure, and finally solvent exchange and drying of the hydrogel to obtain porous nanopapers.

### Preparation of Enzymatic Cellulose Nanofibrils (NFC) Dispersion

The NFC water dispersion was prepared from softwood sulphite pulp fibers (DP of 1200, lignin and hemicellulose contents of 0.7% and 13.8%, respectively, Nordic Pulp and Paper, Sweden). The pulp was first dispersed in water and subjected to a pretreatment step involving enzymatic degradation and mechanical beating. Subsequently, the pretreated pulp was disintegrated by a homogenization process with a Microfluidizer M-110EH (Microfluidics Ind., USA), and a 2 wt % NFC dispersion in water was obtained.

### Preparation of the TO-NFC Dispersion

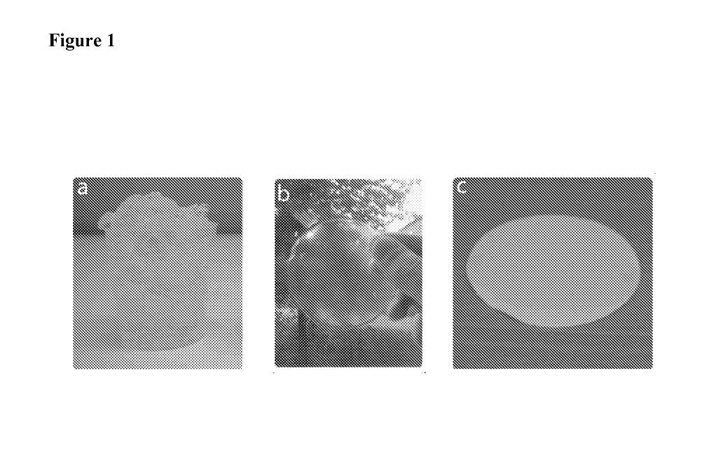

TO-NFC water dispersion was prepared from softwood sulphite pulp fibers (Nordic Pulp and Paper, Sweden). The pulp was first dispersed in water in which sodium bromide and TEMPO were dissolved (1 mmol and 0.1 mmol per gram of cellulose, respectively). The concentration of the pulp in water was 2 wt %. The reaction was started by addition of sodium hypochlorite (10 mmol per gram of cellulose) dropwise into the dispersion. During the addition of NaClO, carboxylate groups were forming on the surface of the fibrils and the pH decreased. The pH of the reaction was then maintained at 10 by sodium hydroxide addition. After all NaClO was consumed, the pulp fibers were filtered and washed several times with deionized water until the filtrate solution was neutral. The purified pulp fibers were then dispersed in water at a concentration of 1 wt % and disintegrated by a homogenization process with a Microfluidizer M-110EH (Microfluidics Ind., USA). A 1 wt % TO-NFC dispersion in water was thus obtained, as shown in FIG. 1a.

Hydrogels from NFC and TO-NFC Dispersions

The NFC or TO-NFC water dispersion (ca 300 mg solid content of cellulose) was diluted to ca 0.1 wt %, degassed and filtrated on top of a 0.65 μm filter nanopaper (DVPP, Millipore) until a strong hydrogel is formed (see picture of the hydrogel in FIG. 1b).

### Preparation of Porous Cellulose Nanopaper

The highly porous cellulose nanopapers were prepared from the NFC hydrogels by three different drying procedures.

### Liquid CO₂ Evaporation (L-CO₂).

The NFC water hydrogel was solvent exchanged to ethanol by first placing it in an ethanol bath (ethanol at 96%) for 24 h and subsequently in the absolute ethanol bath for another 24 h. The NFC ethanol alcogel was then placed in a critical point dryer chamber (Tousimis), the chamber was closed, and liquid carbon dioxide was injected into the chamber under a pressure of ca 50 bars. The sample was kept below the critical point conditions in the chamber to allow solvent exchange from ethanol to liquid CO₂. The chamber was then depressurized and CO₂ evaporated, which led to a porous NFC nanopaper as shown in FIG. 1c.

### Supercritical CO₂ Drying (SC—CO₂).

The NFC alcogel prepared by the above-described procedure was placed in a in a critical point dryer chamber (Tousimis), and liquid carbon dioxide was injected into the chamber under a pressure of ca 50 bars for solvent exchange. The chamber was then brought above the CO₂ critical point conditions to ca 100 bars and 36° C. The chamber was then depressurized and CO₂ evaporated to form a porous NFC nanopaper.

### Tert-Butanol Freeze Drying (Tert-B-FD) for Comparison.

The NFC alcogel is placed in a tert-butanol bath overnight for solvent exchange. It is then freezed by liquid nitrogen (without direct contact of the alcogel with the liquid nitrogen), and the solid tert-butanol is sublimated at room temperature under a vacuum of 0.05 mbar in a benchtop freeze dryer (Labconco Corporation, USA).

### Density and Porosity Measurements

The density of the nanopaper was determined by measuring its weight and dividing it by its volume. The volume was calculated from the thickness of the nanopaper (determined by a digital calliper) and its area. Porosity is deduced from the density of the nanopaper by taking 1460 kg/m³ as density of cellulose¹⁷ using the formula:

$$Porosity = 1 - \frac{{ρ}_{nanapaper}}{{ρ}_{cellulose}}$$

### Specific Surface Area (SSA) and Pore Size Distribution

The Brunauer-Emmett-Teller (BET) surface area was determined by N₂ physisorption using a Micromeritics ASAP 2020 automated system. The porous nanopaper sample was first degassed in the Micromeritics ASAP 2020 at 115° C. for 4 h prior to the analysis followed by N₂ adsorption at −196° C. BET analysis was carried out for a relative vapor pressure of 0.01-0.3 at −196° C. Pore size distribution was determined from N₂ desorption at relative vapor pressure of 0.01-0.99 following a BJH model.

### Field-Emission Scanning Electron Microscopy (FE-SEM)

The in-plane texture of the porous nanopaper was observed by SEM using a Hitachi S-4800 equipped with a cold field emission electron source. The samples were coated with graphite and gold-palladium using Agar HR sputter coaters (ca. 5 nm) Secondary electron detector was used for capturing images at 1 kV.

### Mechanical Properties

Tensile tests of the porous nanopapers were performed using an Instron universal materials testing machine equipped with a 50N load cell. Specimen strips of 30 mm in length and 3-5 mm in width are tested at 10% min⁻¹ strain rate under a controlled relative humidity of 50%. A total of 3 specimens were tested per material. Young's modulus was determined as the slope at low strain, the ultimate strength was determined as the stress at specimen separation. Work to fracture is taken as the area under the stress strain curve.

### Example 1

### Results and Discussion

When the NFC water hydrogel is directly dried, capillary action during water evaporation leads to compaction and a nanopaper of ca. 20% porosity is formed.

Water exchange to methanol or acetone prior to drying increases the porosity to 28% and 40% respectively. This is due to the less hydrophilic character of ethanol and acetone, which reduces capillary effects during drying. In previous work, by Sehaqui, Soft Matter, 2010, 6, 1824-1832, freeze-drying was used to prepare ultra-high porosity foams (porosity of 93-99.5%) from hydrocolloidal NFC dispersions. The NFC materials had a cellular foam structure where the cell wall consisted of aggregated NFC formed during ice crystal growth. The present study aims to preserve the well-dispersed structure of the hydrocolloidal NFC network by alternative drying techniques. First, hydrogels were prepared from NFC and TO-NFC. Water was solvent exchanged into supercritical CO₂, liquid CO₂, and tert-butanol and finally dried using supercritical carbon dioxide drying (SC—CO₂), liquid carbon dioxide evaporation (L-CO₂), and for comparison tert-butanol freeze-drying (Tert-B-FD), respectively.

The NFC nanofibers have a diameter in the 10-40 nm range and no charge on the surface, while the TO-NFC nanofibers have a diameter of 4-5 nm and a carboxylate content of 2.3 mmol/g cellulose. Both NFC and TO-NFC nanofibers have lengths exceeding several micrometres. After filtration, the water volume content in the hydrogel was in the 85-90% range. After exchange of water to ethanol, the TO-NFC alcogel had an ethanol volume content of only about 65%, due to shrinkage of the TO-NFC hydrogel. In contrast, the NFC hydrogel did not show any significant shrinkage during solvent exchange to ethanol (volume content of ethanol in the NFC alcogel is 85-90%). These observations suggest stronger interaction between water and TO-NFC as compared to NFC. This is due to the TO-NFC surface characteristics.

The density and specific surface area of nanopaper materials are summarized in Table 1. The data are related to structural changes during drying. The TO-NFC nanopaper structures have porosities in the 40-56% range, lower than the 74-86% porosity for NFC nanopaper. This is possibly related to the higher charge density on the TO-NFC nanofibers. Supercritical drying of NFC leads to the highest porosity, which is comparable to the ethanol volume in the alcogel prior to drying. Supercritical drying can apparently be performed virtually without shrinkage. The other drying techniques result in nanopaper with lower porosities. Interestingly, NFC nanopaper from the fairly simple liquid CO₂ evaporation route has a porosity as high as 74%. This is much higher than for nanopaper prepared by solvent exchange followed by ethanol or acetone evaporation, where porosities of 28 and 40% resulted. This is due to the low CO₂ polarity, which is in the same range as for toluene, and capillary action is thus reduced compared with ethanol, acetone or water evaporation.

TABLE 1
Density, porosity, estimated fibril diameter based on cylindrical
geometry assumption, average pore diameter from BJH model
and BET specific surface area (SSA) of nanopaper.

| | | | | | Comparison | |
|---|---|---|---|---|---|---|
| | Supercritical CO₂ | | Liquid CO₂ | | tert-butanol | |
| | drying | | evaporation | | freeze-drying | |
| | NFC | TO-NFC | NFC | TO-NFC | NFC | TO-NFC |
| Density | 205 | 640 | 375 | 845 | 380 | 880 |
| (kg/m³) | | | | | | |
| Porosity (%) | 86 | 56 | 74 | 42 | 74 | 40 |
| Specific | 304 | 482 | 262 | 415 | 117 | 45 |
| surface | | | | | | |
| area (m²/g) | | | | | | |
| Average fibril | 9.0 | 5.7 | 10.0 | 6.6 | 23.4 | 60.9 |
| diameter (nm) | | | | | | |
| Average pore | 35.8 | 12.4 | 20.6 | 6.7 | 24.0 | 5.5 |
| diameter (nm) | | | | | | |

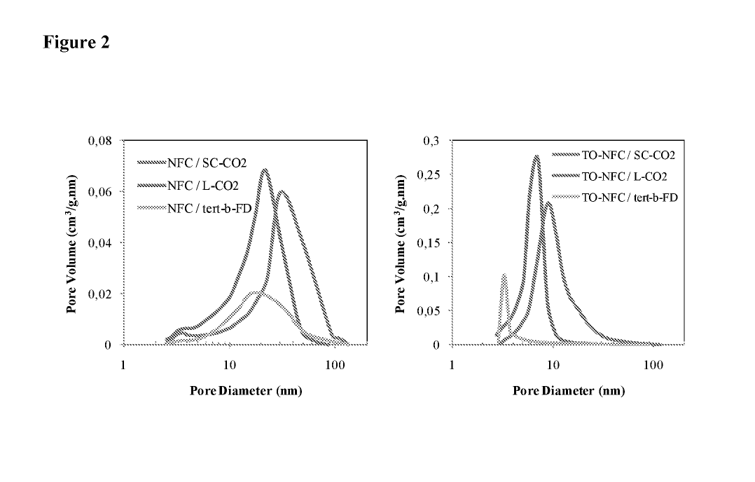

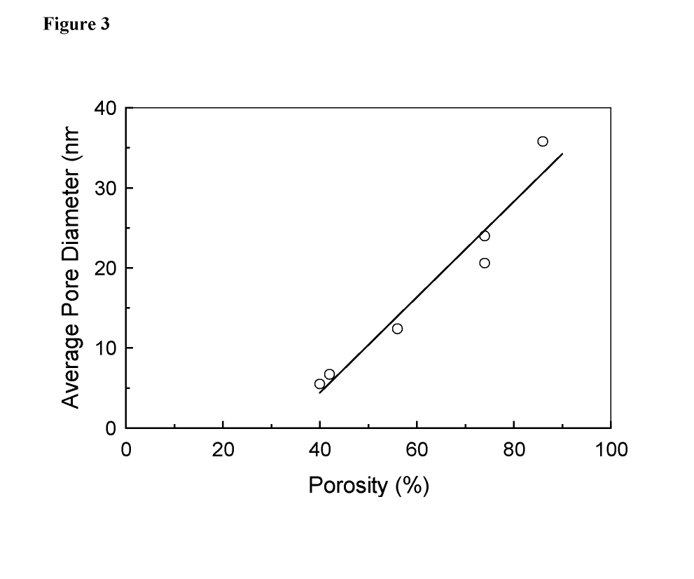

The structure of nanopaper samples was characterized by nitrogen adsorption and scanning electron microscopy. Nitrogen adsorption data are shown in Table 1 and also in FIG. 2 as pore size distribution and FIG. 3 as average BJH pore diameter versus porosity graph. The nanopaper prepared by supercritical drying results in larger BJH pores, which may also be a consequence of the higher porosity. The correlation between porosity and average pore diameter is strong, FIG. 3. The surface area of the NFC nanopaper prepared by SC—CO₂ is 304 m²/g, which is lower than the 482 m²/g of TO-NFC nanopaper. NFC nanopaper also showed lower specific surface area than TO-NFC after nanopaper L-CO₂ preparation. This is due to differences in diameter of the nanofibers, since TO-NFC has a diameter of only around 4 nm, which is smaller than for NFC. The theoretical fibril diameter back-calculated from SSA of the nanopaper, assuming cylindrical nanofiber shape, was 5.7 and 9.0 nm for TO-NFC and NFC respectively. This is agreement with literature data (3-4 nm for TEMPO and 5-20 nm for NFC).

The present nanopaper structures have a nanofiber network structure and the maximum SSA is 482 m²/g, which is the highest SSA reported for native cellulose I NFC materials.

The pore size distribution (FIG. 2) shows that TO-NFC nanopaper has smaller pores than NFC nanopaper. TO-NFC nanopaper is dominated by estimated pore sizes in the 5.5-12.4 nm range, whereas NFC nanopaper is estimated to have most pores in the range 21-36 nm.

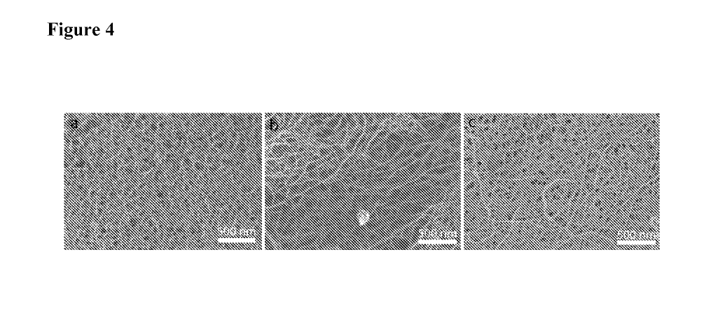

The porous nanopaper structure was investigated by FE-SEM and the results are presented in FIG. 4. NFC nanofibers have a diameter of about 5 nm for TO-NFC and a diameter in the range of 10-30 nm for NFC. The length of the nanofibers is several micrometers. The NFC nanopaper prepared by SC—CO₂ (FIG. 4b, center) appears to have larger pores than TO-NFC prepared by the same method (FIG. 4a, left) in agreement with pore size distribution results. The high SSA nanopaper (TO-NFC, SC—CO₂) in FIG. 4a, left shows a highly homogeneous nanofiber network structure. The nanopaper prepared by Tert-B-FD (FIG. 4c, right) has regions of aggregated NFC although the structural characteristics of an NFC nanofiber network are apparent.

### Mechanical Properties

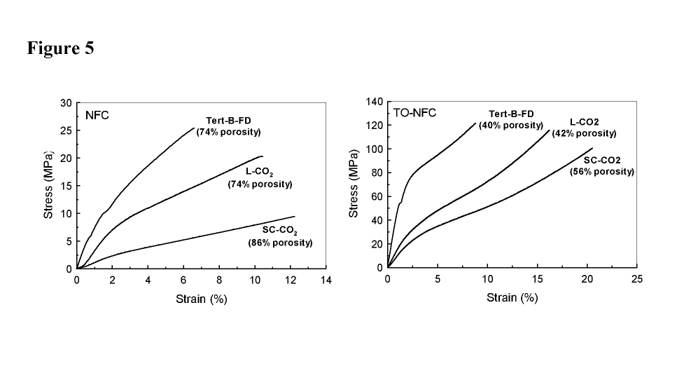

Stress-strain curves and mechanical property data from uniaxial tensile tests are presented in FIG. 5 and Table 2. Higher porosity reduces modulus and strength, as expected. For NFC (left graph), the average strain to failure is in the range 6-10% and the strengths are quite low due to high porosity. The NFC nanopaper with a porosity of 86% has a modulus of 150 MPa and a strength of 7.4 MPa. For the NFC nanopaper prepared by L-CO₂, modulus and strength are 470 MPa and 20 MPa, respectively, at 74% porosity. Interestingly, the NFC nanopaper prepared by tert-butanol freeze-drying had twice the modulus, possibly because of a more agglomerated structure and better bonds between nanofibers in the network. The lower SSA is in support of this hypothesis. Present data may be compared with regenerated cellulose aerogels of 80-90% porosity where moduli are 200-300 MPa and the tensile strength 10-20 MPa. The present cellulose I NFC nanopaper structures of 86% porosity has slightly lower strength and modulus, although the superiority of regenerated cellulose structures in terms of mechanical properties needs to be verified.

TABLE 2
Tensile properties of nanopaper structures based on NFC and TO-
NFC nanofibers. Three different preparation routes, as described
in upper row. Data values in parentheses are standard errors.

| | | | | | Comparison | |
|---|---|---|---|---|---|---|
| | Supercritical CO₂ | | Liquid CO₂ | | tert-butanol | |
| | drying | | evaporation | | freeze-drying | |
| | NFC | TO-NFC | NFC | TO-NFC | NFC | TO-NFC |
| Density | 205 | 640 | 375 | 845 | 380 | 880 |
| (kg m⁻³) | | | | | | |
| Porosity (%) | 86 | 56 | 74 | 42 | 74 | 40 |
| Modulus (GPa) | 0.15 (0.01) | 1.4 (0.2) | 0.47 (0.04) | 1.8 (0.2) | 1.0 (0.01) | 5.0 (0.4) |
| Ultimate | 7.4 (1.8) | 83.7 (15) | 19.6 (1.4) | 102 (12) | 23.2 (2.0) | 120 (3) |
| Strength (MPa) | | | | | | |
| Strain to | 9.6 (2.3) | 16.6 (3.4) | 10.0 (0.7) | 14.7 (1.4) | 5.7 (0.8) | 8.8 (0.4) |
| failure (%) | | | | | | |
| Work to | 0.43 (0.18) | 7.8 (2.7) | 1.2 (0.15) | 8.5 (1.5) | 0.8 (0.2) | 7.3 (0.3) |
| fracture (MJ/m³) | | | | | | |

In FIG. 5, the TO-NFC nanopaper structures (right) show superior mechanical properties, primarily because of higher density. However, the larger strain to failure is interesting as is the soft behavior of the TO-NFC of SSA 482 m²/g prepared by SC—CO₂. In a fiber network model context, the reason for low modulus and low slope for strain-hardening is long segment length between nanofiber-nanofiber bonds. It is interesting to consider the data in Table 2 for TO-NFC nanopaper with 56% porosity; modulus, tensile strength, and strain-to-failure are 1.4 GPa, 84 MPa, and 17%, respectively. These properties are comparable to typical properties for commodity thermoplastics but the density is much lower, 640 kg/m³. The TO-NFC nanopaper structures also have high toughness values for work-to-fracture (area under stress-strain curve). A very interesting application of TO-NFC nanopaper is as nanofiber network reinforcement in nanostructured polymer matrix composites. Possibly, discrete and well-dispersed nanofibers of high content may provide high strain-to-failure in biocomposite structures with ductile matrices.

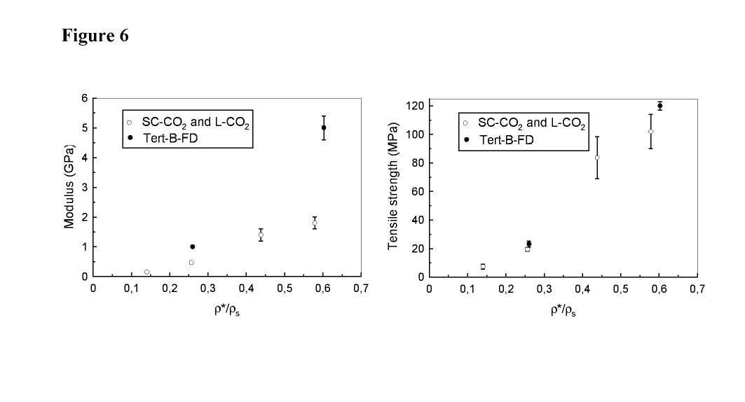

In FIG. 6, Young's modulus in tension and tensile strength are presented as a function of relative density (ratio between nanopaper density and cellulose density). Tensile strength scales almost linearly with the relative density. There seems to be no strong effect from preparation route or specific surface area (for SSA, see Table 1). In contrast, the Tert-B-FD preparation route has strong effects and much higher modulus at high relative density. Previous nanopaper structures prepared from ethanol and acetone evaporation with around 40% porosity have moduli in the 7-9 GPa range. The TO-NFC nanopaper prepared by L-CO₂ has a modulus of 1.8 GPa. To explain this (and the Tert-B-FD observation) one may consider fiber network models with fiber aspect ratio between fiber-fiber bonding sites as an important parameter for network stiffness predictions. In preparation routes with low specific surface area (Tert-B-FD), the fiber aspect ratio between fiber-fiber bonding sites is lowered and modulus is increased. Thus, the present preparation routes provide increased control of nanofiber network structures and the corresponding deformation behavior.

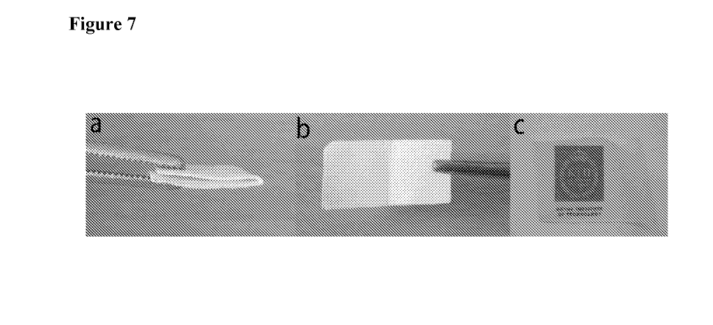

The investigated nanopaper structures were flexible (low modulus and high strain-to-failure) and durable in repeated bending, as illustrated in FIG. 7, similar to what has been described for aerogels. 180° folding is easily performed with low force (a) and no apparent fracture events are visible even after 10 cycles of folding-unfolding (b). This reflects the small diameter of NFC nanofibers in combination with high NFC strength. A simple model for the minimum radius of curvature ρₘᵢₙ a fiber can sustain before fracture is:

ρₘᵢₙ=Ed/2σ_{f}

where E is Young's modulus, d fiber diameter and σ_{f} is fiber strength. A reduction in fiber diameter from roughly 10 μm of conventional microfibers to 10 nm of the present nanofibers is therefore very significant. It will have a dramatic effect on the minimum radius of curvature of a fiber which is bent in a fiber network structure.

Also, the TO-NFC nanopaper prepared by SC—CO₂ is presented in FIG. 7 c), where its optical transparency is apparent, despite a porosity of 42%. This also indicates that the present nanopaper structures have a low extent of nanofiber aggregation. Water-dried nanopaper structures can also be transparent or translucent, but have a much lower specific surface area.

### Example 2

### Materials

NFC dispersion based on enzymatic pretreatment of the wood pulp (NFC) was prepared from softwood sulphite pulp fibers (DP of 1200, lignin and hemicelluloses contents of 0.7% and 13.8%, respectively, Nordic Pulp and Paper, Sweden). The pulp was first dispersed in water and subjected to mechanical beating followed by pretreatment using endoglucanase enzymes. Subsequently, the enzyme-treated pulp was disintegrated in a homogenization process using a Microfluidizer M-110EH (Microfluidics Ind., USA). A 2 wt % NFC dispersion in water was obtained.

NFC dispersions based on TEMPO-oxidation pretreatment (TO-NFC) were prepared from the same softwood sulphite pulp fibers. The pulp was first dispersed in water in which sodium bromide and TEMPO (2,2,6,6-tetramethylpiperidine-1-oxy radial) were dissolved (1 mmol and 0.1 mmol per gram of cellulose respectively). The concentration of the pulp in water was 2 wt %. The reaction was started by adding sodium hypochlorite (NaClO) dropwise to the dispersion (5 mmol per gram of cellulose). Throughout the addition of NaClO, negative charge (carboxylate groups) was introduced on the surface of the cellulose fibrils and the pH decreases. pH of the reaction was then maintained at 10 by adding NaOH solution. After all NaClO is consumed, the pulp fibres were filtered and washed several times with deionized water until it became white. The TEMPO-treated pulp was dispersed in water at a concentration of 1 wt % and was then disintegrated by one pass through a Microfluidizer M-110EH (Microfluidics Ind., USA). A 1 wt % NFC dispersion in water was obtained.

### Aerogel Preparation

An aqueous NFC dispersion was mixed with about twice its volume of tert-butanol using an Ultra Turrax mixer (IKA, D125 Basic) during 10 minutes. The obtained mixture was subjected to centrifugation, and the supernatant fraction was removed. The lower fraction of the dispersion was used, stirred, placed in a cup (15 mm in height and 50 mm in diameter), and frozen using liquid nitrogen. The frozen liquid is sublimated overnight in a FreeZone 6 liter benchtop freeze dryer (Labconco Corporation, USA) at a sublimation temperature of −53° C. and a pressure of 0.05 mbar, to form a NFC aerogel having a density of ca 15 kg/m³.

As an alternative, solvent exchange of the NFC dispersion from water to ethanol was carried out in three steps, followed by solvent exchange from ethanol to tert-butanol in three steps. Ethanol or tert-butanol was added, mixed and subjected to centrifugation, and the supernatant fraction was removed. At the last step, freezing and sublimation of the solvent were done as described for the 1-step solvent exchange and resulted in low density aerogel samples (14 and 29 kg/m³ density).

High density aerogel samples were prepared from high concentration NFC water dispersions placed in a cup, and then placed over night in large excess of 1) ethanol at 96%, 2) pure ethanol and 3) pure tert-butanol. Samples were then frozen and sublimated as previously described to produce higher density aerogel samples (50 and 105 kg/m³).

### Field-Emission Scanning Electron Microscopy (FE-SEM)

Prior to micro-structural analysis, all samples were dried overnight in a dessicator filled with silica gel. Fractured surfaces were observed. The specimens were fixed on a metal stub using carbon tape and coated with a double-layer coating consisting of graphite and gold-palladium using Agar HR sputter coaters. A Hitachi S-4800 scanning electron microscope operated at 1 kV was used to capture secondary electron images of aerogel cross-sections.

### Specific Surface Area and Pore Size Distribution by Nitrogen Adsorption

The Brunauer-Emmett-Teller specific surface area (BET) was determined by N₂ physisorption using a Micromeritics ASAP 2020 automated system. 0.1-0.2 g of aerogel sample was first degassed in the Micromeritics ASAP 2020 at 115° C. for 4 hrs prior to the analysis followed by N₂ adsorption at −196° C. BET analysis was carried out for a relative vapor pressure of 0.01-0.3 at −196° C. From the experimental BET specific surface area values (BET), the corresponding diameter of the fibril d in the aerogel was estimated from equation 1 assuming a cylindrical shape of the fibrils and assuming that the density of cellulose ρ_{c} is equal to 1460 kg m⁻³. The average pore size of the NFC aerogels was estimated from the nitrogen desorption isotherm according to the analysis of Barrett-Joyner-Halendar (BJH).

$$d = \frac{4}{{ρ}_{c} \cdot BET} \qquad ( 1 )$$

### Density and Porosity

The density of the aerogels (ρ*) was estimated by dividing their weight by their volume as measured by a digital caliper. Their porosity was calculated from equation 2 where the ratio ρ*/ρ_{c} is the relative density.

$$Porosity = 1 - \frac{{ρ}^{*}}{{ρ}_{c}} \qquad ( 2 )$$

### Compression Test

Aerogel samples having a cylindrical shape of 2 cm in diameter and 15 mm in height were compressed in a Miniature Materials Tester (MiniMat2000) equipped with a load cell of 20, 200 or 2000 N (depending on aerogel density) at a strain rate of 1.5 mm·min⁻¹. The modulus was calculated from the initial linear region of the stress-strain curves, the energy absorption is defined as the area below the stress-strain curve from 0 to 70% strain. From stress-strain curves, energy absorption diagrams (energy absorption vs stress diagrams) were plotted. Each stress value was related to the energy absorbed up to this stress (i.e. to the area below the stress-strain curve).

### Example 2

### Results

### Nitrogen Adsorption

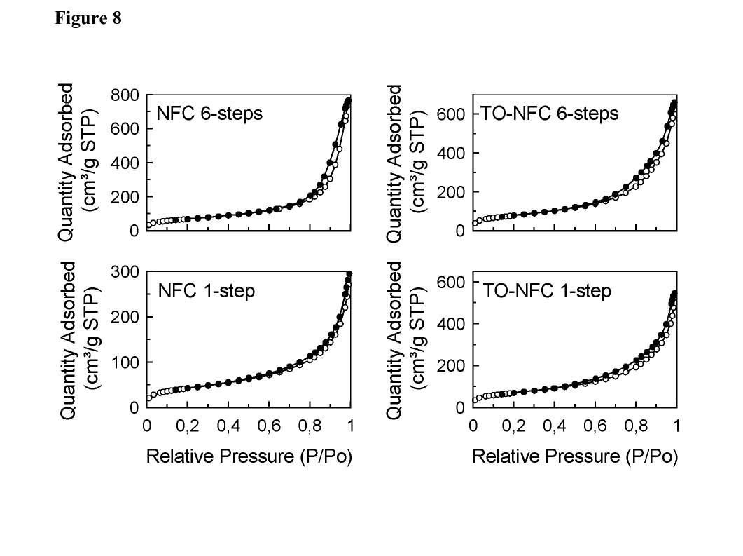

Nitrogen adsorption was used to estimate specific surface area and porosity characteristics of the NFC aerogels. Sorption isotherms are presented in FIG. 8 for NFC aerogels prepared by 1-step and 6-steps solvent exchange. According to the IUPAC classification, all the sorption isotherms are of type IV which involves adsorption on mesoporous adsorbents with strong adsorbate-adsorbent interaction.

The specific surface area is an important structural characteristic of aerogels. High values are desirable for applications such as functional carriers (e.g., catalysis, fuel storage, drug release), and electrical applications such as electrodes. Nyström et al showed that the high surface area of cellulose from Cladophora algae (80 m²/g) can be used for making ultrafast paper batteries by coating the fibrils with a conductive polymer. In the present study, the inventors aimed at preserving the surface area of native wood cellulose NFC dispersions through first solvent exchange from water to tert-butanol followed by rapid freeze-drying. The specific surface area of the aerogels was determined from the adsorption isotherms in FIG. 8 at relative pressures below 0.3 using BET analysis. From the BET specific surface area values, the corresponding diameter of the fibrils in the aerogel was estimated assuming they have cylindrical shape. These results are presented in Table 3.

TABLE 3
Specific surface area of NFC aerogels and estimated average nanofibrils
diameter based on an assumption of cylindrical shape of nanofibrils.

| | Specific surface area | Average fibril diameter |
|---|---|---|
| | (m²/g) | (nm) |
| NFC, 1-step | 153 | 17.9 |
| NFC, 6-steps | 249 | 11.0 |
| TO-NFC, 1-step | 254 | 10.8 |
| TO-NFC, 6-steps | 284 | 9.6 |

Results presented in Table 3 show that the present aerogels have high specific surface area with a maximum of 284 m²/g. This reflects the small diameter of the fibrils and the effectiveness of the drying method. The favourable dispersion of the NFC in water is largely preserved also in the dried state. Aerogels prepared according to the 1-step method have a lower specific surface area than aerogels prepared according to the 6-steps method. This is expected since about 30% water is present in the 1-step hydrogel after exchange to tert-butanol. A larger extent of NFC aggregation takes place during freeze-drying when water is present. In a previous study, even rapid freeze-drying of aqueous NFC dispersions resulted in a significant aggregation of NFC and a cellular foam structure was formed with “nanopaper” cell walls.

During water freezing, ice crystals nucleate, grow and push NFC to interstitial regions between crystals so that aggregated NFC nanopaper cell walls are formed. It is likely that additional aggregation takes place during the sublimation phase. This results in NFC foams having a relatively low specific surface area (14-42 m²/g). The present results demonstrate the advantage by tert-butanol freeze-drying for surface area preservation. Ishida et al reported significant difference between aerogels from tunicate whiskers prepared by regular freeze-drying (25 m²/g) and tert-butanol solvent exchange (130 m²/g).

The surface area difference between NFC and TO-NFC aerogels prepared by 6-steps solvent exchange is not as large as expected. This could be due to some aggregation in the TO-NFC aerogel reflected in a theoretical diameter of the fibrils of 9.6 nm which is about double the diameter reported for TO-NFC. The theoretical diameter of 6-steps NFC is 11 nm, it suggests very limited aggregation of the fibrils. In a technical perspective it is interesting to note the high specific surface area of the TO-NFC aerogel prepared by a simple 1-step solvent exchange (254 m²/g).

The BJH model has been widely used to estimate pore size distribution for cellulose aerogels. The physical significance of the estimated distribution is unclear since the real pores may have different geometries than assumed. This model was used to assess the difference in the average pore size between different aerogels. Table 4 presents the average pore diameter obtained from desorption isotherms in FIG. 8 according to BJH analysis. The 1-step methods seem to result in smaller pore size. The estimated average pore diameter of the present NFC aerogels are comparable to those previously reported for regenerated cellulose aerogels prepared by carbon dioxide supercritical drying (average pore diameter of 7-39 nm and 9-12 nm respectively). It should be noted however, that these estimates are based on certain assumptions which have not been verified, and in addition, large pores are not included in gas adsorption measurements. The estimated pore diameters are therefore primarily indicative measures.

TABLE 4
Average pore diameter of the NFC aerogels calculated from BJH analysis.
Porosity of the aerogels is between 98.5 and 99%.

| | NFC, | | | TO-NFC, |
|---|---|---|---|---|
| | 1-step | NFC, 6-steps | TO-NFC, 1-step | 6-steps |
| Average pore | 11.2 | 16.0 | 10.9 | 11.8 |
| diameter/nm | | | | |

BJH analysis is not sensitive to large pore sizes due to the limitation of gas sorption experiments. Porosity characteristics of NFC were also studied using field emission scanning electron microscope (FE-SEM).

### FE-SEM

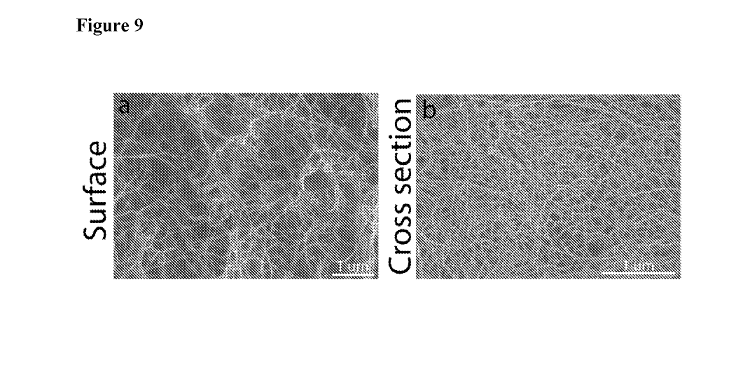

FIG. 9 shows micrographs of a surface (a) and a cross-section (b) of NFC aerogel prepared by 6-steps solvent exchange with a density of 30 kg/m³. No sign of an ice-templated cellular foam structure and no significant NFC aggregation were observed, and a fibrillar network structure represented the NFC aerogels. The NFC-NFC joints in the network are apparent. NFC diameters are estimated to be in the range 10-40 nm, but could be smaller since a coating was applied to the aerogel before SEM observation. The pores present on the surface (a) are larger than those present in the aerogel bulk (b) and pores are sub-micrometric. Differences in pore size between the surface and cross section of the aerogels were also observed by others and could be due to temperature gradients through-the-thickness during freezing. This may result in a certain pore size distribution. The present NFC aerogel mainly has pore dimensions well below 1 micrometer.

### Mechanical Properties in Compression

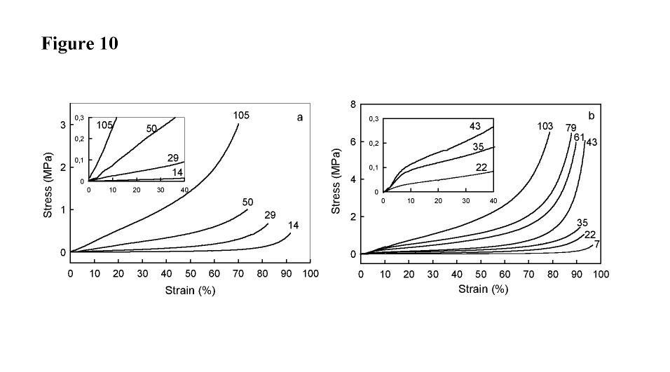

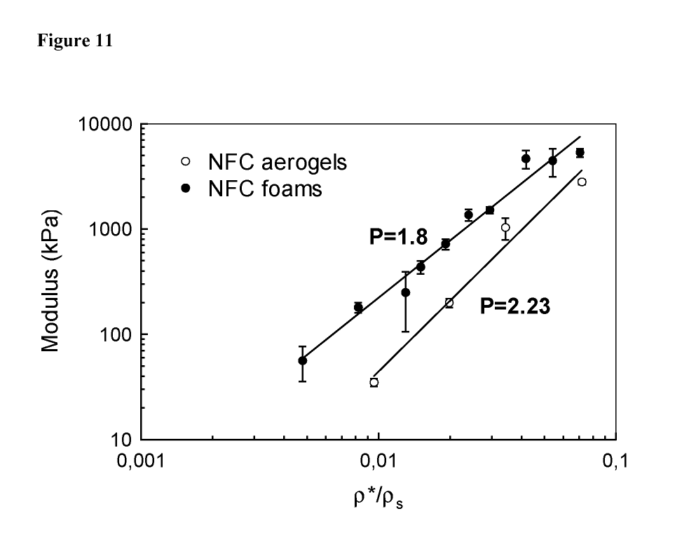

The mechanical properties of NFC aerogels with 4 different densities (14, 29, 50, 105 kg/m³) were investigated. Stress-strain curves of the aerogels are presented in FIG. 10 together with stress-strain curves of NFC foams. Mechanical property data of NFC aerogels are summarized in Table 5 and modulus comparisons are made to NFC foams and presented in FIG. 11.

TABLE 5
Mechanical properties in compression of NFC aerogels. Values within
parentheses represent standard deviations.

| | Density (kg/m³) | | | |
|---|---|---|---|---|
| | 14 | 29 | 50 | 105 |
| Porosity (%) | 99.0 | 98.0 | 96.6 | 92.8 |
| Modulus (kPa) | 34.9 (3.0) | 199 (19) | 1030 (240) | 2796 (155) |
| Strength (kPa) | 3.2 (0.4) | 24.4 (3.9) | 69 (8) | 238 (21) |
| Energy absorption | 10.8 (0.8) | 68 (1) | 223* | 720 (20) |
| (kJ/m³) | | | | |

*only one sample reached 70% strain

The present NFC network aerogels exhibit ductile behaviour and can be compressed to large strains (>80%), see FIG. 11. NFC fibrils deform primarily by bending as fibers typically do in low density fiber networks. The ductility is because individual NFC nanofibers can bend to very small radius of curvature. This in turn is a consequence of the small diameter of NFC nanofibers in combination with high NFC strength. In contrast, inorganic aerogels are typically very brittle.

The stress-strain curves in show a strain-hardening behaviour already at low strain, and no yield stress can be detected. The relationship between NFC aerogel modulus E* and relative density is E*˜(ρ*/ρₛ) (see figure caption in FIG. 11 for definition of relative density). Please note that the meaning of the term “modulus” is unclear since the stress-strain relationship is linear to large strains although the material is truly elastic (recovers after deformation) only at strains below 3%. The proportionality factor 2.2 is much lower than the corresponding relationship for silica aerogel modulus which is E*˜(ρ*/ρₛ). The strong modulus dependence on density for silica aerogels has been explained by a highly heterogeneous network at low density. Loose “dangling” structural members are present for silica aerogels, which do not contribute to modulus. It seems likely that NFC network aerogels are much more homogeneous in structure and carry load efficiently also at ultra-high porosity (FIG. 10.a). Compared to NFC foams, the modulus of the present NFC network aerogels depends more strongly on relative density (slope 2.2 compared with 1.8 for NFC foams). NFC aerogel modulus is lower than modulus of NFC foams, particularly at low densities. Although a closed cell foam is stiffer than an open cell foam at the same relative density detailed reasons for the observed difference in FIG. 11 are unclear.

A previously proposed modelling approach for the prediction of elastic modulus of a 3D fiber network is used in the present study for the estimation of the L/D ratio of fiber segments between joints in the NFC network. The equations for the prediction of the elastic modulus of a network of cylindrical fibers having a joint-to-joint length of L and a fiber diameter of D is:

$$\frac{{E}^{*}}{{E}_{f}} = \frac{9. f}{32. {( L / D )}^{2}} \qquad ( 3 )$$

E_{f} is the modulus of cellulose fibrils, two different values for E_{f} are used for L/D calculation, namely 32 GPa and 84 GPa, f is NFC volume fraction (equal to ρ*/ρₛ), and E* is the experimental value of the NFC aerogel modulus. Values of L/D estimated from equation 3 are presented in Table 6.

TABLE 6
L/D estimation of NFC aerogel networks of different densities

NFC aerogel density

| | 14 | 29 | 50 | 105 |
|---|---|---|---|---|
| L/D (if E_{f} = 32 GPa) | 49 | 30 | 17 | 15 |
| L/D (if E_{f} = 84 GPa) | 79 | 48 | 28 | 24 |

In the context of the model, the increased E* with increasing density can be interpreted as if the NFC nanofiber “beams” in the network become shorter and therefore stiffer as the fiber volume fraction f is increasing (f=relative density ρ*/ρ). In the case of an NFC aerogel of 29 kg/m³ density (porosity=98%) the L/D becomes 30-48. The NFC nanofiber diameter back-calculated from the surface area of 6-step NFC aerogels is 10 nm The joint-to-joint length L would then be 300-480 nm according. Looking at the FE-SEM micrographs in FIG. 10, L was estimated to be in the range 100-1000 nm The assumed geometry of the fiber network model and the model itself thus seem realistic and helpful in the development of our understanding of NFC nanofiber aerogels.

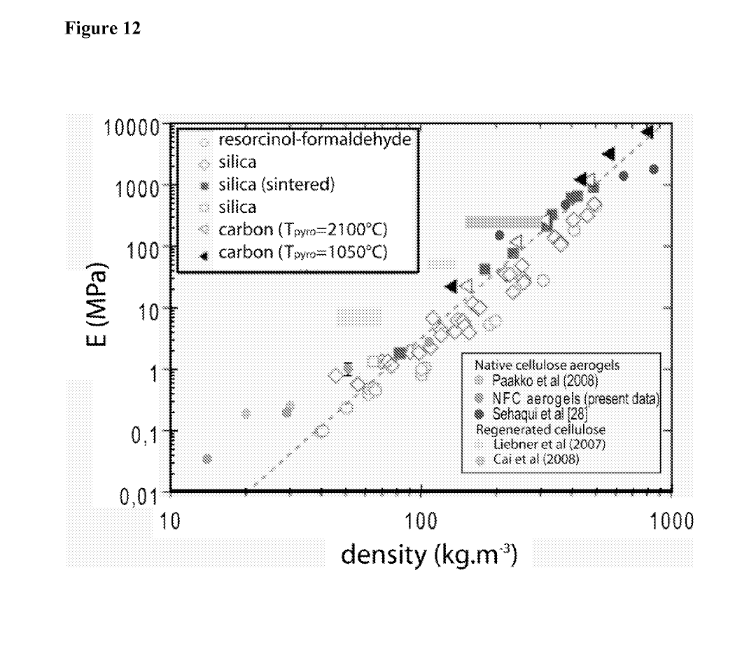

In FIG. 12, moduli for many different types of aerogels are plotted versus density. Data for the present NFC aerogels are included as well as data for other cellulose aerogels collected from the literature. It is interesting to notice the high modulus offered by cellulose aerogels compared to other types of aerogels when data are related to density. One may also note that the present aerogels occupy a previously empty property space at low densities. The few data in the literature for regenerated cellulose aerogels (dissolved cellulose which is precipitated in a liquid as cellulose II) indicate their superior properties compared to NFC aerogels, although more data are needed to verify this. The densities for regenerated cellulose aerogels reported in the literature tend to be higher than for the present NFC.

### Energy Absorption

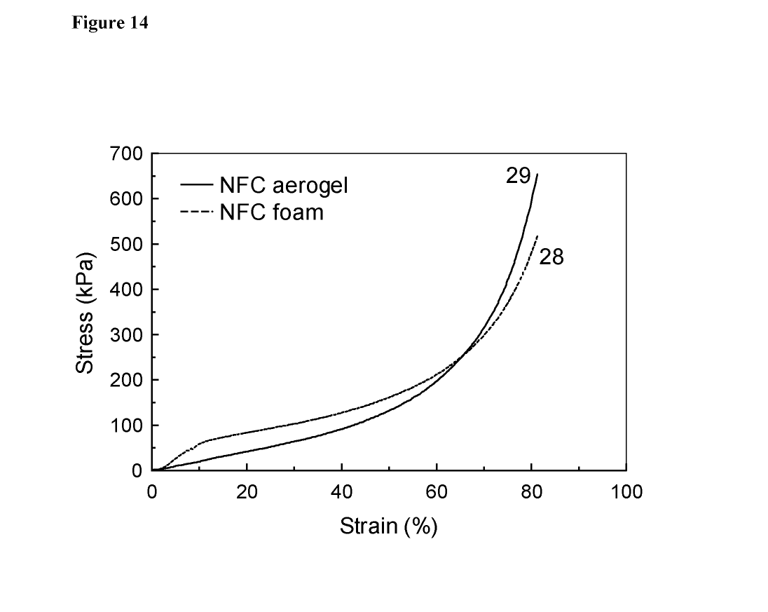

Energy absorption characteristics of the present aerogels are investigated and quantified as the area under stress-strain curves up to 70% strain. Energy absorption is relevant in applications such as packaging, where the material absorbs energy as it collapses under compression. The results for energy absorption in 13 show that the energy absorption of the present aerogels compare well with that of closed cell synthetic polymer foams used for packaging such as expanded polystyrene, and to previously reported NFC foams, and are far better than clay aerogels. For example, a 50 kg/m³ NFC aerogel has an energy absorption of 223 kJ/m³, comparable to the energy absorptions of a 55 kg/m³ polystyrene foam (250 kJ/m³) and a 43 kg/m³ NFC foam (210 kJ/m³), and greater than the energy absorption of a clay aerogel of 52 kg/m³ (23 kJ/m³). The linear strain hardening behaviour due to NFC nanofiber bending is different from what is observed for polymer foams that show a flat collapse region in which the stress is constant with increasing the strain. There is also a difference between NFC aerogels and NFC foams, see FIG. 14. The NFC aerogel is softer at lower strains, but then reaches higher stress at high strains. Reasons are to be found in different deformation mechanisms for NFC nanofiber networks and NFC foams. The present preparation routes allow a wide density range and a variety of structures (NFC aerogels or foams) so that materials can be tailored for applications such as packaging and protection.

1. A cellulose-based material comprising nanofibrillated cellulose (NFC) from native cellulose, characterized in that the material has a specific surface area (SSA) of at least 200 m²/g and a nanofiber network structure, wherein the nanofibers have a diameter less than 40 nm.

2. The material according to claim 1, wherein the native cellulose is cellulose of type I.

3. The material according to claim 1, wherein the material has a specific surface area of at least 300 m²/g.

4. The material according to claim 1, wherein the material has a specific surface area of at least 400 m²/g.

5. The material according to claim 1, wherein the nanofibers have a diameter in the range from 2 to 20 nm.

6. The material according to claim 1, wherein the nanofibers have a diameter in the range from 3 to 10 nm.

7. The material according to claim 1, wherein the cellulose material is a nanoporous solid, an aerogel, a nanopaper, and/or a membrane.

8. A method for preparing a cellulose-based material from native cellulose according to claim 1, said cellulose-based material comprising nanofibrillated cellulose (NFC) in the form of cellulose type I, said method comprising the following steps of:
(a) obtaining a hydrogel comprising NFC in the form of cellulose type I;
(b) substantially exchanging the solvent of the NFC dispersion at least once for at least one second solvent; and,
(c) removing the at least one second solvent by at least one of (i) liquid evaporation and (ii) supercritical drying.

9. The method according to claim 8, wherein the dispersion of step (a) is an aqueous dispersion.

10. The method according to claim 8, wherein the at least one second solvent is a water-miscible solvent.

11. The method according to claim 8, said method comprising the following steps of:
(a) obtaining a hydrogel comprising NFC in the form of cellulose type I by disintegrating native cellulose;
(b) obtaining an organogel by substantially exchanging the solvent of the hydrogel of step (a) at least once for at least one water-miscible solvent; and,
(c) removing the at least one water-miscible solvent by at least one of (i) liquid evaporation and (ii) supercritical drying.

12. The method according to claim 8, wherein step (a) comprises enzymatic treatment and/or mechanical treatment and/or chemical treatment.

13. The method according to claim 12, wherein the enzymatic treatment comprises endoglucanase treatment, exoglucanase treatment, and/or cellulase treatment.

14. The method according to claim 12, wherein the chemical treatment comprises 2,2,6,6-tetramethylpiperidine-1-oxy radical (TEMPO) oxidation, carboxymethylation, acid treatment, and/or base treatment.

15. The method according to claim 8, wherein step (a) can be divided into the following sub-steps:
(a1) disintegrating native cellulose into NFC in the form of cellulose type I; and
(a2) filtrating the NFC of step (a1) to obtain a hydrogel comprising NFC in the form of cellulose type I.

16. The method according to claim 8, wherein step (b) can be divided into the following sub-steps:
(b1) substantially exchanging the solvent of the hydrogel of step (a) for a water-miscible organic solvent; and
(b2) mixing the water-miscible organic solvent with CO₂.

17. The method according to claim 15, wherein CO₂ is present in liquid form.

18. The method according to claim 7, wherein the liquid evaporation step (i) and/or the supercritical drying step (ii) are preceded by step (b) comprising substantially exchanging the solvent of the hydrogel of step (a) for a water-miscible organic solvent followed by substantially exchanging the water-miscible organic solvent for CO₂.

19. The method according to claim 7, wherein the cellulose-based material is a nanoporous solid, an aerogel, a nanopaper, and/or a membrane.

20. A cellulose-based material obtainable through the method of claim 7.

21. Use of the cellulose-based material according to claim 1 as a nanoporous solid, an aerogel, a nanopaper, and/or a membrane.

22. The cellulose-based material according to claim 1, wherein the cellulose-based material has a porosity of at least 20%.

23. The cellulose-based material according to claim 1, wherein the cellulose-based material has a modulus of at least 0.1 GPa.

24. The cellulose-based material according to claim 1, wherein the cellulose-based material has an ultimate strength of at least 0.5 MPa.

25. The cellulose-based material according to claim 1, wherein the cellulose-based material has a strain-to-failure of at least 1%.

26. The cellulose-based material according to claim 1, wherein the cellulose-based material has an average pore diameter of at least 1 nm.
